(19) United States
(12) Patent Application Publication
(10) Pub. No.: US 2012/0178990 A1
(43) Pub. Date: Jul. 12, 2012

(54) Phallus Retention Apparatuses

(75) Inventor: Cole Astin, Los Angeles, CA (US)

(73) Assignee: BODY DESIGNS WEST, INC., Henderson, NV (US)

(21) Appl. No.: 13/497,531

(22) PCT Filed: Sep. 21, 2010
(86) PCT No.: PCT/US2010/049729

§ 371 Date: Mar. 21, 2012

Related U.S. Application Data
(60) Provisional application No. 61/244,081, filed on Sep. 21, 2009.

(51) Int. Cl. A61F 5/00

(52) U.S. Cl. 600/41

(57) ABSTRACT

A phallus retention apparatus for retaining a prosthetic phallus relative to a hand of a wearer with a sheath, a thumb aperture in the sheath, and a prosthetic phallus aperture either directly in the sheath or in a retention flap coupled to the sheath for receiving a shaft portion of a prosthetic phallus. The phallus aperture is spaced from the thumb aperture to position the phallus aperture approximately centered on the palmar side of the sheath. A phallus retention apparatus can retain a prosthetic phallus relative to an external structure with a base panel, a retention panel, and a phallus aperture in the retention panel for receiving the body portion of the phallus therethrough.

### TECHNICAL FIELD

The present invention relates generally to apparatuses for retaining prosthetic phalluses. More particularly, disclosed and protected herein are embodiments of phallus retention apparatuses for enabling the effective and comfortable retention of a prosthetic phallus and potentially other stimulation aids in relation to the body of a wearer and, potentially, relative to other structures, such as a chair.

### BACKGROUND OF THE INVENTION

The prior art has disclosed numerous structures for retaining a phallus relative to a wearer's body and relative to other structures. For example, various harnesses have been disclosed for retaining a phallus relative to the pelvis of a wearer. Other structures have been disclosed for retaining a phallus in relation to inanimate objects.

Unfortunately, the harnesses and other arrangements of the prior art suffer from a number of limitations and disadvantages. By way of example, a review of commercially available harnesses will make clear that they are typically formed with a rigid crotch plate that is retained relative to a wearer by a similarly rigid belt arrangement. For example, in U.S. Pat. No. 4,488,541, Garcia disclosed what is referred to as a Therapeutic Adapter wherein a pubic shield has a plurality of conical projections and a monolithically formed tubular portion. The pubic shield and the tubular portion are held in place by a system of waist and leg belts. In a similar vein, a Sexual Aid is disclosed in U.S. Pat. No. 5,103,810 to Chang wherein a tubular body is retained by a guard member and a connection piece. The device is retained in place by a waistband and bands coupled to the connection piece.

The structures taught by Garcia, Chang, and many other disclosures of the prior art can thus be seen to be quite rigid and unnatural in structure and application. Such prior art harnesses can be uncomfortable and restrictive to the wearer. Moreover, harnesses of the prior art can be uncomfortable for the further reason that the retained phallus, which normally must be particularly designed for each harness, has a proximal end that directly contacts the wearer's body. Still further, the devices of the prior art have been generally limited in their application to being applied to a wearer's pelvis. As a result, many devices of the prior art are incapable of being retained relative to other parts of the wearer's body or relative to external structures.

The present inventor improved upon the previous state of the art with the retention arrangements disclosed in U.S. Pat. No. 6,849,041 for a Phallus Retention Harness. There, a phallus retention harness was disclosed with a pocket member with front and rear panels that enabled a prosthetic phallus to be retained relative to a user in a comfortable yet effective manner not only during sexual encounters but also as during normal daily activities in an inconspicuous manner under a wearer's clothing if so desired. Other embodiments of the invention of the '041 patent enable a phallus to be worn relative to a wearer's hand or other body part.

The invention of the '041 patent achieved a number of advantages and benefits beyond the structures and methods of the prior art. Nonetheless, the present inventor has appreciated that even further advantages could be realized through the creation of phallus retention apparatuses that can be comfortably worn or otherwise retained that provide enhanced capabilities and alternative applications even as compared to the present inventor's own previous, advantageous developments. Indeed, particularly in light of the many less than ideal structures taught by the prior art and the deficiencies left by those structures as appreciated by the present inventor, it is clear that the development of improved phallus retention apparatuses capable of retaining phalluses in a comfortable and effective manner relative to varied portions of a wearer's body and, potentially, relative to external structures would represent a useful advance in the art.

### SUMMARY DISCLOSURE OF THE INVENTION

Advantageously, the present invention is founded on the basic object of providing phallus retention apparatuses that enable a phallus to be retained relative to a user in a comfortable yet effective manner.

A further object of certain embodiments of the invention is to provide phallus retention apparatuses that enable a phallus to be retained relative to varied locations on a wearer's body.

Another object of particular embodiments of the invention is to provide a phallus retention apparatus that can enable a phallus to be retained relative to an external structure, such as a chair, in an effective manner.

Yet another object of the invention is to provide a phallus retention apparatus that enables a phallus to be removed and replaced relative to the phallus retention apparatus in a simple and convenient manner.

A further object of the invention is to provide phallus retention apparatuses that prevent inadvertent dislodging of a retained phallus.

Another object of embodiments of the invention is provide a phallus retention apparatus that can additionally retain complementary stimulus aids, such as vibratory devices, in proximity to a retained phallus.

These and in all likelihood further objects and advantages of the present invention will become obvious not only to one who reviews the present specification and drawings but also to those who have an opportunity to make use of an embodiment of the phallus retention apparatuses disclosed herein. However, it will be appreciated that, although the accomplishment of each of the foregoing objects in a single embodiment of the invention may be possible and indeed preferred, not all embodiments will seek or need to accomplish each and every potential advantage and function. Nonetheless, all such embodiments should be considered within the scope of the present invention.

In carrying forth the objects set forth above, one embodiment of the phallus retention apparatus is constructed to retain a prosthetic phallus relative to a hand of a wearer. The phallus retention apparatus has a sheath of flexible material that has a first end and a second end. At least the first end is open for receiving the hand of the wearer from a first direction. A thumb aperture is disposed in the sheath through which a thumb of the wearer can be passed. A prosthetic phallus aperture for receiving a shaft portion of a prosthetic phallus is retained relative to the sheath spaced from the thumb aperture.

In one practice of the invention, the material of the sheath can be substantially continuous but for the thumb aperture. Further, in certain embodiments, the second end of the sheath can be open for receiving the hand of the wearer from a second direction whereby the phallus retention apparatus can be retained relative to the left or right hand of the wearer.

The phallus aperture can be disposed directly in the sheath. Where the sheath is considered to have a palmar side and a dorsal side, the phallus aperture can be spaced from the thumb aperture to position the phallus aperture approximately centered on the palmar side of the sheath and approximately perpendicular to the thumb aperture. Additionally, the phallus aperture and the thumb aperture can be generally aligned with one another laterally in relation to the sheath.

Where desirable to adjust the effective size of the sheath to accommodate hands of different sizes, the phallus retention apparatus can further include at least one cinching strap laterally retained relative to the sheath in combination with a means for cinching the cinching strap about the sheath. Sleeves can be fixedly or adjustably coupled to the sheath adjacent to the phallus aperture for selectively retaining stimulus aids, such as vibratory devices. In certain embodiments, an extension panel can have a proximal end retained by the sheath and at least one finger loop can be retained by a distal end of the extension panel.

Under certain embodiments, the phallus retention apparatus can include a retention flap with a proximal end fixed to the sheath, an elongate body portion with a distal end, and a means for selectively securing the retention flap wrapped at least partially around the sheath. The phallus aperture can then be disposed in the retention flap. The proximal end of the retention flap can, for example, be fixed to the sheath adjacent to the palmar side of the thumb aperture. Additionally, the phallus aperture can be spaced from the thumb aperture to position the phallus aperture approximately centered on the palmar side of the sheath and approximately perpendicular to the thumb aperture when the retention flap is wrapped at least partially around the sheath. Particular embodiments of the phallus retention apparatus can also include at least one flexible ring with a central aperture for being passed over the shaft portion of the prosthetic phallus.

In other embodiments, the phallus retention apparatus can be constructed to retain a prosthetic phallus relative to an external structure, such as, but not limited to, a thigh of a wearer or a seat. Such an arrangement can be founded on a base panel, a retention panel, and a means for coupling the retention panel to the base panel. A phallus aperture can be disposed in the retention panel for receiving the body portion of the phallus therethrough. Finally, means can be provided for retaining the base panel and the retention panel in relation to the external structure, whether it be the thigh of a user, a chair, or some other structure separate from the phallus retention arrangement.

The means for fastening the retention panel to the base panel could, for example, comprise mechanical fastening tape that could entirely surround the resilient phallus aperture. Furthermore, at least one sleeve can be provided together with means for selectively retaining the at least one sleeve adjacent to the phallus aperture for selectively retaining a stimulus aid, such as a vibratory device. The means for selectively retaining the at least one sleeve adjacent to the phallus aperture could take the form of mechanical fastening tape.

The means for retaining the base panel and the retention panel in relation to the external structure could comprise a means for selectively coupling a first end portion of the base panel to a second end portion of the base panel to surround at least a portion of the external structure, such as a means for directly coupling the first and second end portions of the base panel. Alternatively, the means could comprise at least one strap and means for securing the at least one strap to surround at least a portion of the external structure. The retention panel and the base panel could be entirely separable, or they could have portions, such as their ends, secured together at what may be considered a hinge connection.

One will appreciate that the foregoing discussion broadly outlines the more important features of the invention to enable a better understanding of the detailed description that follows and to instill a better appreciation of the inventor's contribution to the art. Before any particular embodiment or aspect thereof is explained in detail, it must be made clear that the following details of construction and illustrations of inventive concepts are mere examples of the many possible manifestations of the invention.

### BRIEF DESCRIPTION OF THE DRAWINGS

In the accompanying drawings:

FIG. 1 is a perspective view of a phallus retention apparatus for retaining a prosthetic phallus relative to a wearer's hand in an open configuration;

FIG. 2 is a top plan view of the phallus retention apparatus of FIG. 1, again in an open configuration;

FIG. 3 is a bottom plan view of the phallus retention apparatus of FIG. 1 in an open configuration;

FIG. 4 is a view in front elevation of the phallus retention apparatus of FIG. 1 in an open configuration;

FIG. 5 is a view in rear elevation of the phallus retention apparatus of FIG. 1, again in an open configuration, with a phallus inserted therein;

FIG. 6 is a view in right side elevation of the phallus retention apparatus of FIG. 1 in an open configuration;

FIG. 7 is a view in left side elevation of the phallus retention apparatus of FIG. 1 in an open configuration;

FIG. 8 is a perspective view of the phallus retention apparatus of FIG. 1 shown retaining a prosthetic phallus relative to a hand of a wearer;

FIG. 9 is a perspective view of an alternative phallus retention apparatus for retaining a prosthetic phallus relative to a hand of a wearer;

FIG. 10 is a view in right side elevation of the phallus retention apparatus of FIG. 9;

FIG. 11 is a view in left side elevation of the phallus retention apparatus of FIG. 9;

FIG. 12 is a top plan view of the phallus retention apparatus of FIG. 9;

FIG. 13 is a bottom plan view of the phallus retention apparatus of FIG. 9;

FIG. 14 is a view in front elevation of the phallus retention apparatus of FIG. 9;

FIG. 15 is a view in rear elevation of the phallus retention apparatus of FIG. 9;

FIG. 16 is a perspective view of the phallus retention apparatus of FIG. 9 shown retaining a prosthetic phallus relative to a hand of a wearer;

FIG. 17 is a perspective view of a phallus retention apparatus for retaining a phallus relative to an external structure, such as a chair;

FIG. 18 is a bottom plan view of the phallus retention apparatus of FIG. 17;

FIG. 19 is a bottom plan view of the phallus retention apparatus of FIG. 17 in a dismantled configuration;

FIG. 20 is a perspective view of the phallus retention apparatus of FIG. 17 retaining a phallus and vibratory devices relative to a chair;

FIG. 21 is a perspective view of an alternative phallus retention apparatus for retaining a prosthetic phallus relative to an extremity of a wearer or other structure;

FIG. 22 is a bottom plan view of the phallus retention apparatus of FIG. 21;

FIGS. 23 and 24 are top plan and perspective views of the phallus retention apparatus of FIG. 21 in an opened configuration; and

FIG. 25 is a perspective view of the phallus retention apparatus of FIG. 21 retaining a prosthetic phallus relative to a thigh of a wearer.

### DETAILED DESCRIPTION

The present invention for phallus retention apparatuses is subject to a wide variety of embodiments. However, to ensure that one skilled in the art will be able to understand and, in appropriate cases, practice the present invention, certain preferred embodiments of the broader invention revealed herein are described below and shown in the accompanying drawing figures.

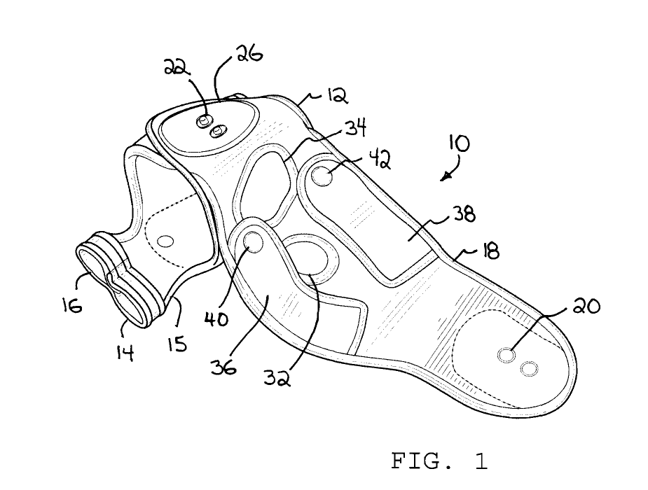

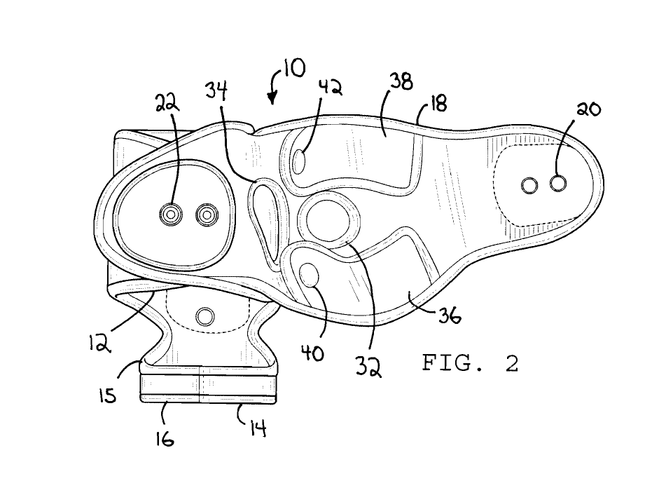

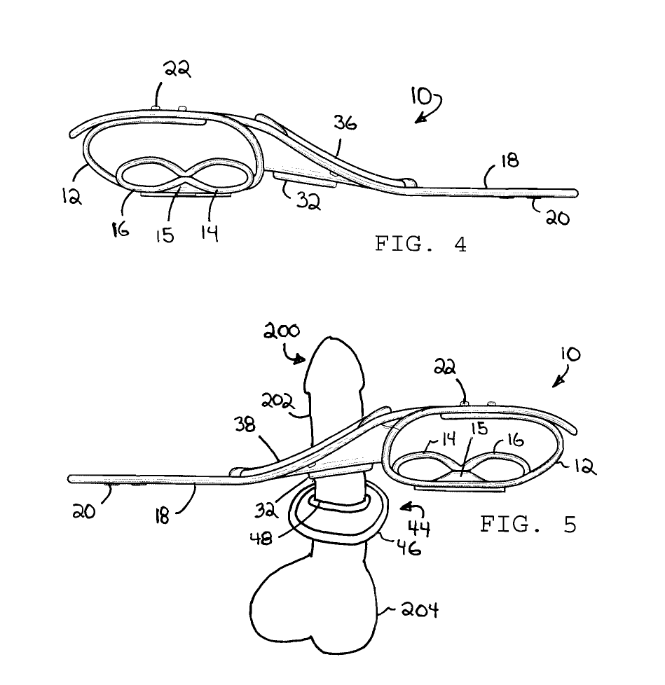

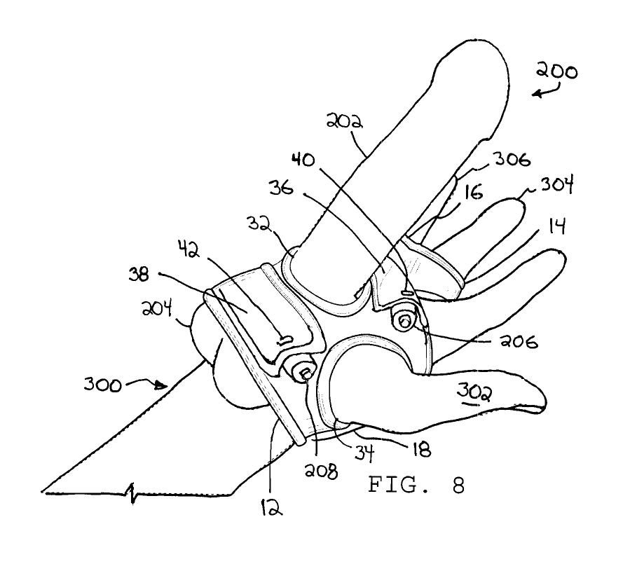

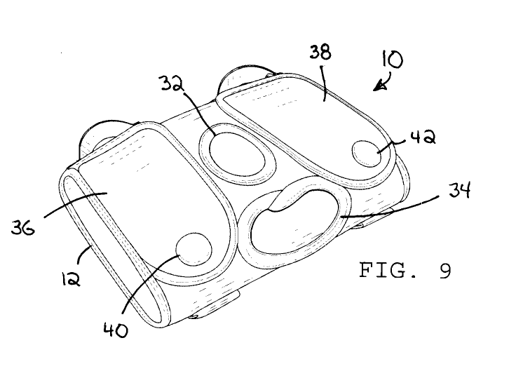

A first phallus retention apparatus according to the present invention is indicated generally at 10 in FIGS. 1 through 8. There, the phallus retention apparatus 10 has a sheath 12 of flexible material for receiving a hand 300 of a wearer as is shown in FIG. 8. The sheath 12 has a thumb aperture 34 through which a thumb 302 of the wearer can be passed. The material of the sheath 12 is substantially continuous but for the open thumb aperture 34. An extension panel 15 extends longitudinally from the palm of the sheath 12. First and second finger loops 14 and 16 are disposed at the distal end of the extension panel 15 for receiving first and second fingers 304 and 306 of the wearer. With this, the phallus retention apparatus 10 can be securely retained relative to a hand 300 of a wearer while leaving the wearer's thumb 302 and fingers 304 and 306 substantially free.

A retention flap 18 has a proximal end fixed to the sheath 12 and an elongate body portion with a distal end. In this example, the proximal end is fixed to the sheath 12 adjacent to a lower or palmar side of the thumb aperture 34. The body portion of the retention flap 18 has a length sufficient to allow it to wrap around the sheath 12 until the distal end thereof reaches adjacent to an upper or dorsal side of the thumb aperture 34.

The retention flap 18 can be secured in position wrapped around the sheath 12 by any suitable means. In the depicted embodiment, fastening means include mating snap button fasteners 20 and 22 that are provided on the inner surface of the distal end of the retention flap 18 and on the outer surface of the sheath 12 adjacent to the thumb aperture 34. Also provided are mating sections 24 and 26 of hook and loop material on the inner surface of the distal end of the retention flap 18 and on the outer surface of the sheath adjacent to the thumb aperture 34 for further or alternatively retaining the retention flap 18 in position wrapped around the sheath 12.

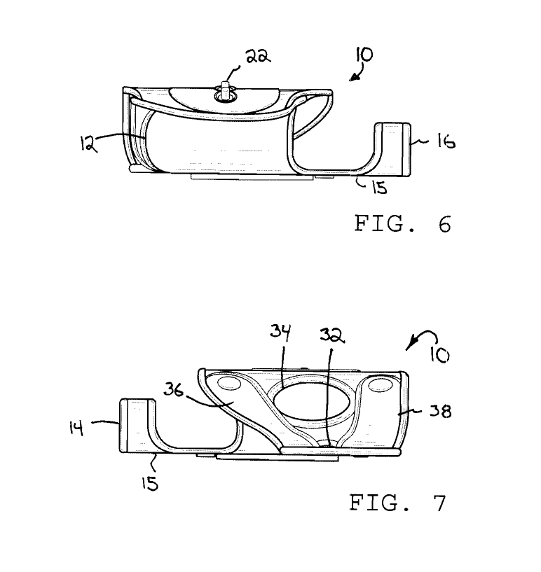

The retention flap 18 also has a prosthetic phallus aperture 32 for receiving a shaft portion 202 of a prosthetic phallus 200 as shown, for example, in FIGS. 5 and 8. The phallus aperture 32 is spaced from the thumb aperture 34 by a distance sufficient to place the phallus aperture 32 generally at the center of the palm of the hand 300 of a wearer when the retention flap 18 is disposed to wrap around the sheath 12 as shown in FIG. 8. When the phallus retention apparatus 10 is positioned on a wearer's hand 300 as in FIG. 8, the phallus aperture 32 is disposed approximately perpendicular to the thumb aperture 34. The phallus aperture 32 and the thumb aperture 34 are roughly aligned laterally.

The depicted embodiment of the phallus retention apparatus 10 is designed for use on the right hand 300 of a wearer. Of course, the phallus retention apparatus 10 could readily be oppositely configured for use on the left hand 300 of a wearer.

Under this arrangement, a phallus 200 having a shaft portion 202 and a base portion 204 can be engaged with the phallus retention apparatus 10 by passing the shaft portion 202 through the phallus aperture 32 until the base portion 204 of the phallus 200 reaches the phallus aperture 32. The peripheral ring defining the phallus aperture 32 is resiliently flexible whereby prosthetic phalluses 200 of varying shapes and sizes can be received and retained.

Where necessary to prevent the base 204 of a phallus 200 from inadvertently passing through the phallus aperture 32 or otherwise to adjust the interrelationship between the phallus 200 and the phallus retention apparatus 10, one or more rings 44 with a flexible body portion 46 and an aperture 48 defined by a resiliently flexible ring can be passed over the shaft portion 202 effectively to build up, supplement, or even partially or entirely form the base portion 204. The use of such rings 44 may only be required with prosthetic phalluses 200 having base portions 204 that are sufficiently small that a risk of passing through the phallus aperture 32 is presented. Embodiments of the invention are also contemplated where rings or other panels of material, which can be padded, can be selectively interposed between the base portion 204 of the phallus 200 and the sheath 12 to cushion the hand 300 of the wearer as may be necessary or helpful, for example, with phalluses 200 having narrower base portions 204 and non-resilient phalluses 200.

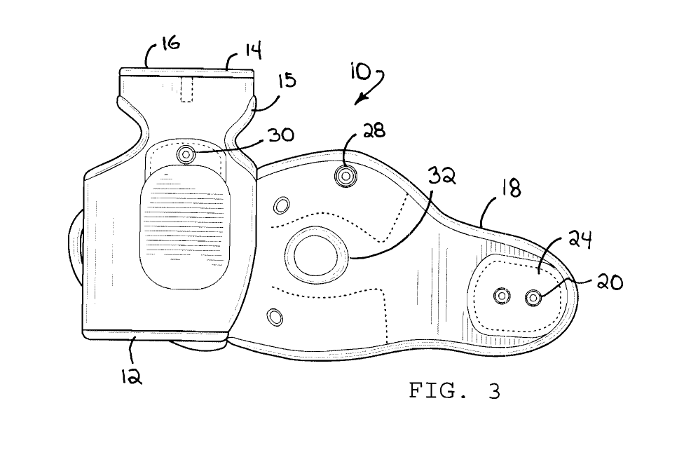

To ensure further that the phallus 200 does not become inadvertently dislodged, the retention flap 18 can have additional means for being secured to the sheath 12 in the area of the phallus aperture 32 and thus adjacent to the base portion 204 of the phallus 200. In the present embodiment, for example, an additional snap fastener combination 28 and 30, which is seen best in FIG. 3, is provided on the inner surface of the retention flap 18 and the outer surface of the sheath 12 to the palmar side of the sheath 12 and on the facing portion of the retention flap 18. With this, the snap fastener combination 28 and 30 can be secured once the phallus 200 is in place to prevent the phallus 200 from becoming inadvertently dislodged.

The phallus retention apparatus 10 also provides means for retaining additional stimulus aids, in this case one or more vibratory devices 206 and 208, adjacent to the phallus aperture 32 and thus adjacent to the base portion 204 of the phallus 200. More particularly, in the depicted embodiment, first and second pockets or sleeves 36 and 38 are provided to opposite sides of the aperture 32. With this, first and second vibratory devices 206 and 208 or other stimulus aids can be inserted into the first and second sleeves 36 and 38 respectively. Closure arrangements 40 and 42, which in this embodiment comprise snap buttons, are provided for selectively closing off the first and second sleeves 36 and 38 to retain the vibratory devices 206 and 208 in place. The sleeves 36 and 38 can be fixed in place as by sewing, or the sleeves 36 and 38 could be removable and repositionable, such as by mechanical fastening tape or another effective means.

It will be appreciated that the materials employed to craft the phallus retention apparatus 10 could vary depending on a number of factors, including market goals, user preferences, and other circumstances. In one presently preferred embodiment, the sheath 12, the extension panel 15, the retention flap 18, and potentially other components of the phallus retention apparatus 10 can be formed from a resilient rubber, preferably neoprene synthetic rubber, chosen for its durability, ability to be washed, comfort, resiliency, and other factors.

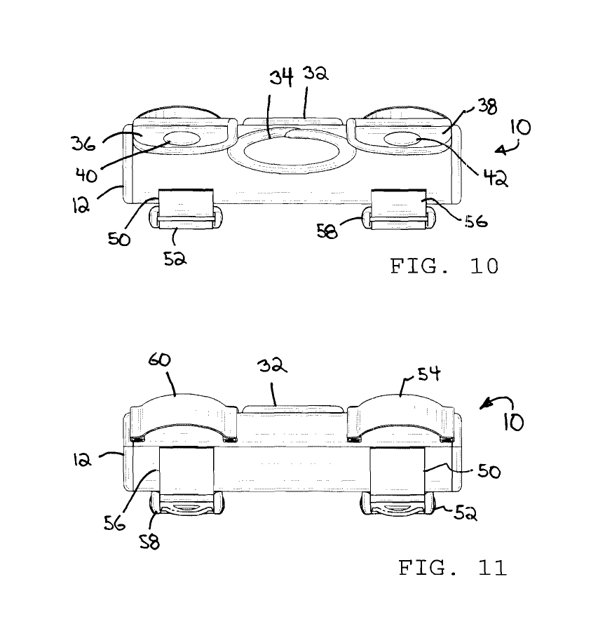

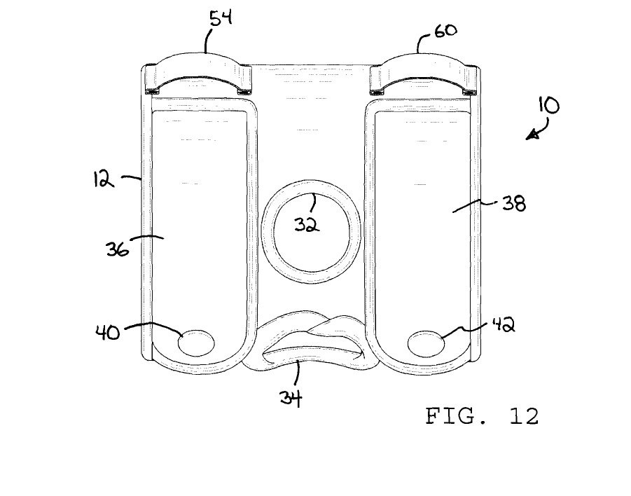

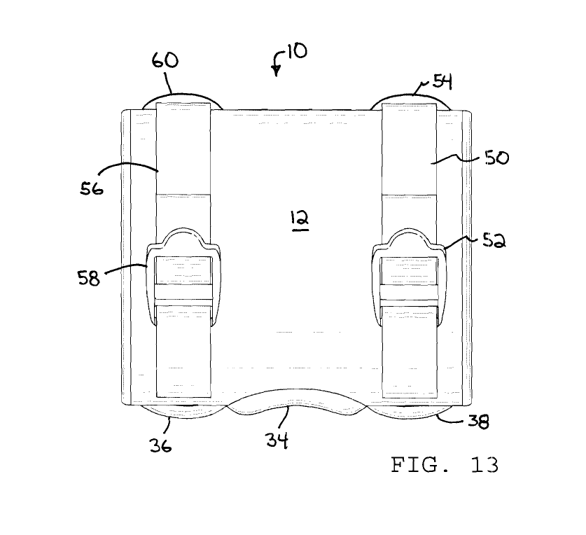

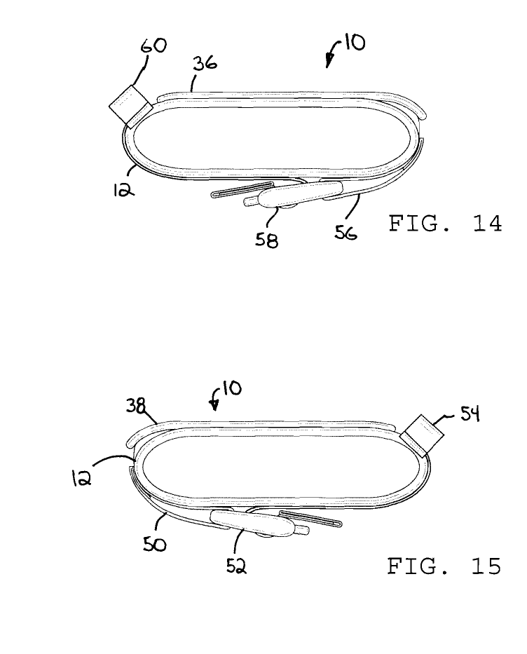

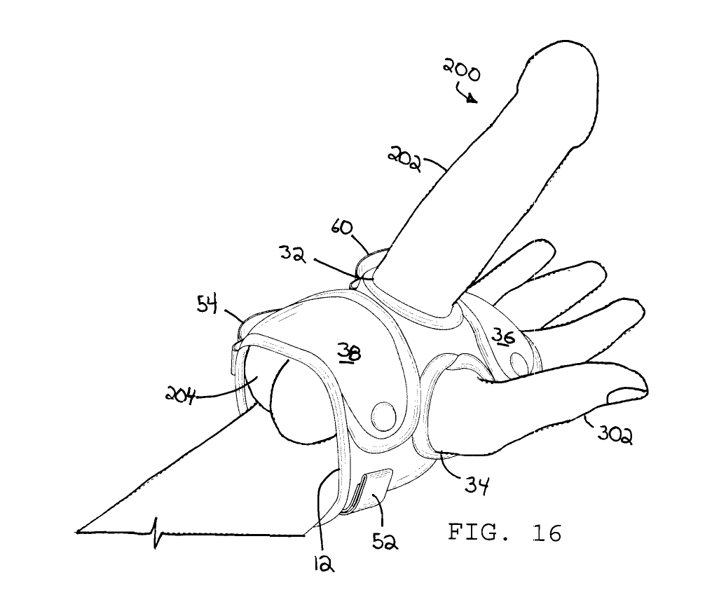

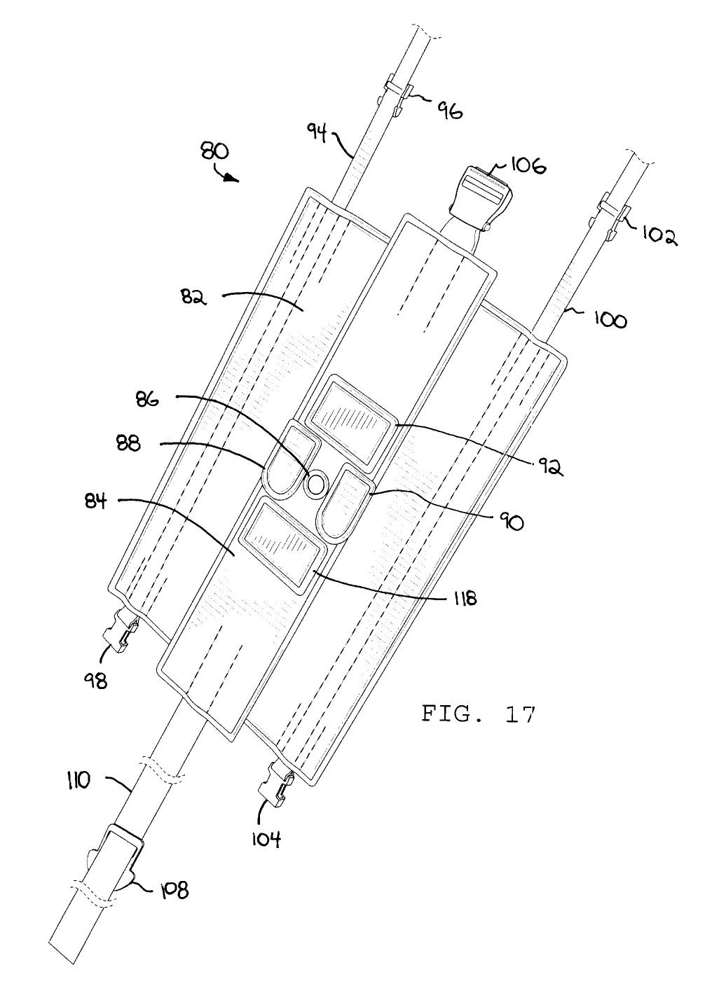

An alternative phallus retention apparatus 10 for retaining a prosthetic phallus 200 in relation to a hand 300 of a wearer is shown in FIGS. 9 through 16. There, the phallus retention apparatus 10 again has a sheath 12 designed to receive the hand 300 of a wearer. A thumb aperture 34 is disposed in the sheath 12 for receiving a thumb 302 therethrough. In this embodiment, however, the sheath 12 has the phallus aperture 32 disposed directly therein, and the retention flap 18 is foregone. The phallus aperture 32 is spaced from the thumb aperture 34 to position the phallus aperture 32, and thus a retained prosthetic phallus 200, approximately centered on the palm of the hand 300 as shown in FIG. 16. When the phallus retention apparatus 10 is positioned on a wearer's hand 300 as in FIG. 16, the phallus aperture 32 is disposed approximately perpendicular to the thumb aperture 34. The phallus aperture 32 and the thumb aperture 34 are again generally aligned laterally.

Advantageously, the phallus retention apparatus 10 in this embodiment can be applied to either the left or the right hand 300 of a wearer with no change in structure. Where a user wishes to apply the phallus retention apparatus 10 to his or her right hand 300, he or she can insert his hand into the sheath 12 from a first end thereof with the phallus aperture 32 aligned with his or her palm. This would be the left end of the sheath in, for example, FIG. 9. When the hand 300 is so inserted, the arrangement shown in FIG. 16 is achieved. However, where a user wishes to apply the same phallus retention apparatus 10 to his or her left hand, the user need only insert his or her hand from the second, opposite end of the sheath 12, again with the phallus aperture 32 aligned with his or her palm.

First and second cinching straps 50 and 56 are provided adjacent to the first and second ends of the sheath 12 for adjusting the effective size of the sheath 12 to enable an accommodation of users of different hand sizes. The straps 50 and 56 have slide buckles 52 and 58 engaged therewith for enabling a selective fastening of the straps 50 and 56, and first and second loops 54 and 60 are disposed to align with the first and second straps 50 and 56 respectively to receive excess lengths of the straps 50 and 56 where necessary. Again, first and second sleeves 36 and 38 are provided to opposite sides of the phallus aperture 32 for receiving vibratory devices 206 and 208. The sleeves 36 and 38 can again be closed by snap closure combinations 40 and 42. The sheath 12 and the sleeves 36 and 38 can again be formed from any suitable material, including neoprene rubber.

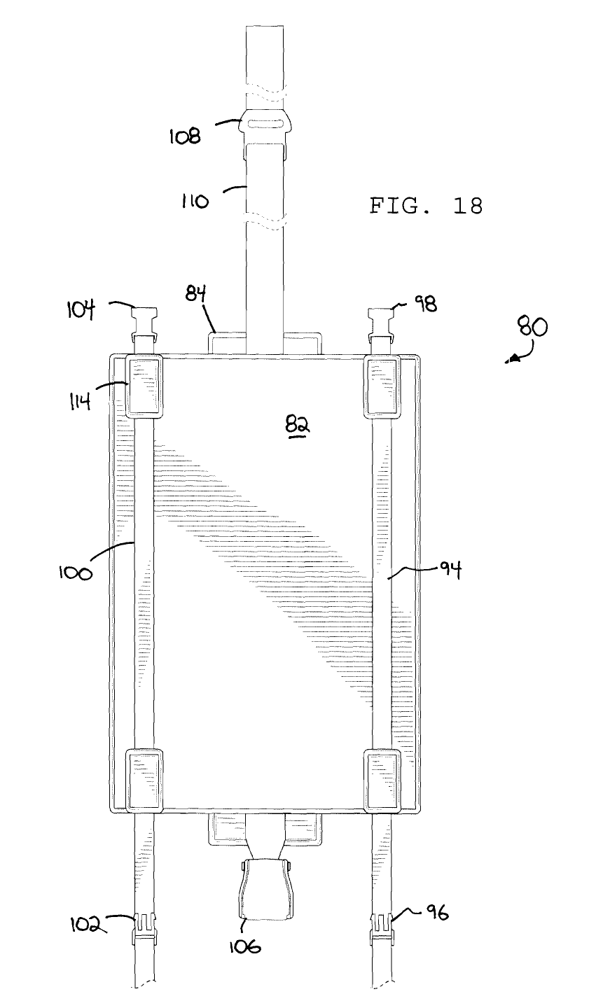

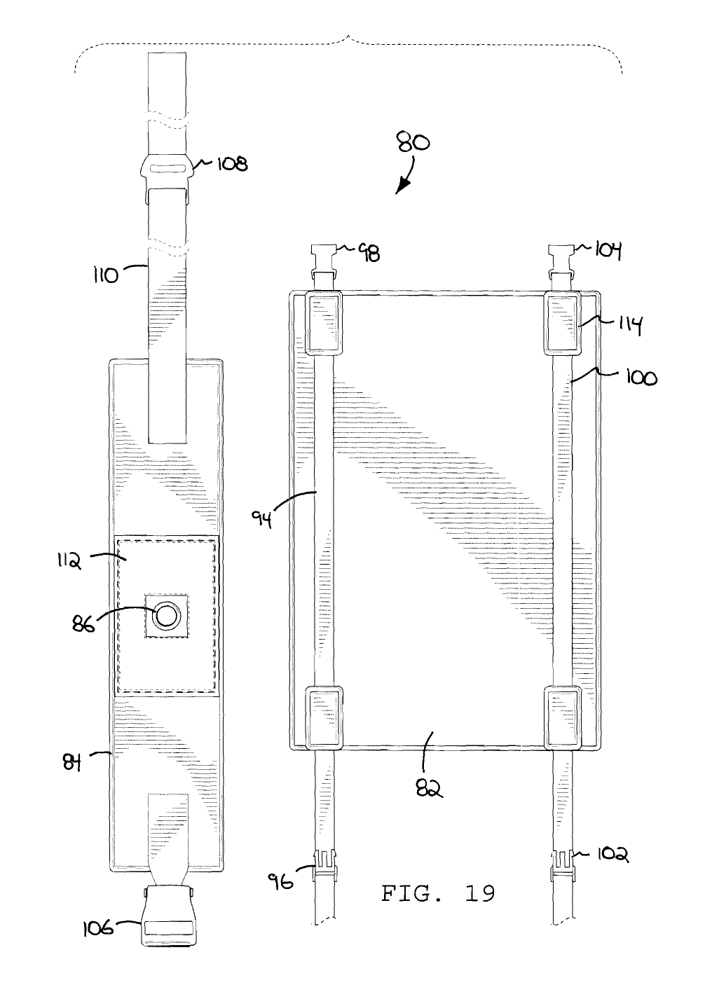

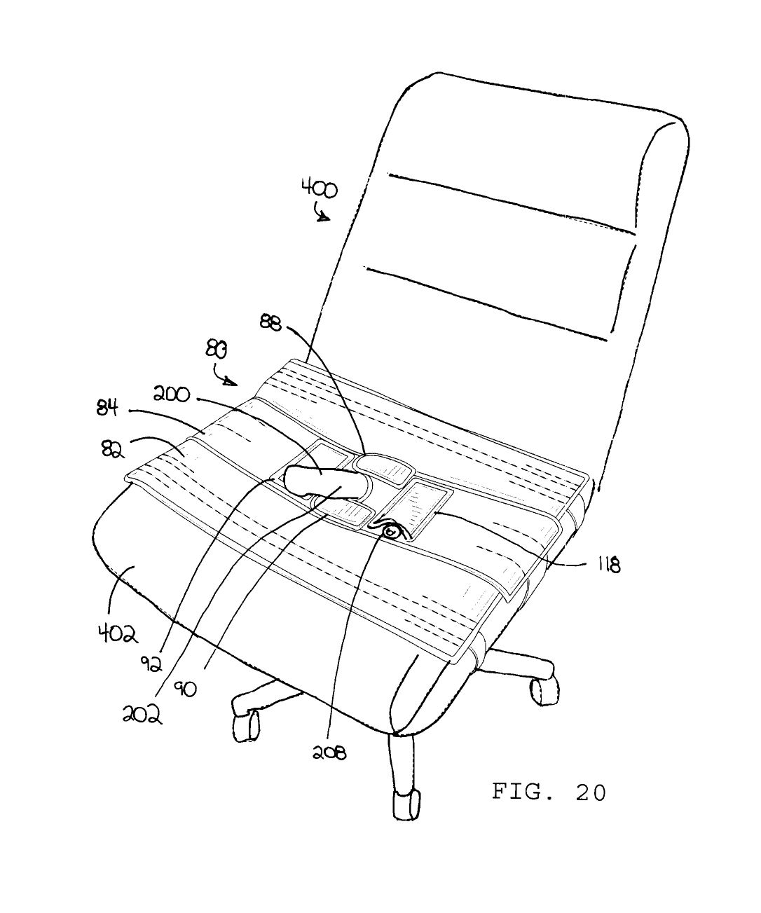

Another variation of the invention is shown in FIGS. 17 through 20. There, the phallus retention apparatus is indicated generally at 80 and is adapted to be retained relative to an external structure, in this case a chair 400. The phallus retention apparatus 80 has a flexible base panel 82 sized to cover a substantial portion of a seat 402 of a chair 400. A retention panel 84 is removably coupled to the base panel 82 by any suitable means, such as mechanical fastening tape, hook and loop combinations, or any other effective arrangement. The retention panel 84 has a phallus aperture 86 for receiving the body portion of a phallus 200 received therethrough as shown in FIG. 20. In this example, the means for fastening retention panel 84 to the base panel 82 comprises mechanical fastening tape 112 that entirely surrounds the resilient phallus aperture 86. The mechanical fastening tape 112 thereby secures the retention panel 84 in place in an adjustable location and orientation relative to the base panel 82. So disposed, the fastening tape 112 additionally acts to prevent inadvertent movement and dislodging of a retained prosthetic phallus 200 that is inserted through the phallus aperture 86.

Sleeves 88, 90, 92, and 118 are provided for receiving vibratory devices 206 and 208 or other stimulus aids. The sleeves 88, 90, 92, and 118 are removably and replaceably retained relative to the retention panel 84 by fastening means 116 and 120 comprising mechanical fastening tape or similar adjustable and removable and replaceable means so that the number, orientation, and location of the sleeves 88, 90, 92, and 118 can be adjusted. Sleeves 88, 90, 92, and 118 and the stimulus aids retained thereby thus can be readily added, removed, and repositioned as desired.

The base panel 82 has first and second longitudinally disposed straps 94 and 100 retained by sleeves 114 to the reverse side of the base panel 82. Slidable buckle members 96 and 102 are disposed on the straps 94 and 100 for engaging corresponding buckle members 98 and 104 that are fixed to the opposite ends of the straps 94 and 100. The buckle members 96 and 102 are disposed to a first end of the base panel 82, and the buckle members 98 and 104 are disposed to a second, opposite end of the base panel 82. With this, the base panel 82 can be secured to a chair 400 or other structure as shown in FIG. 20 by wrapping the straps 94 and 100 around the seat portion 402 of the chair 400, engaging the buckle members 96, 98, 102, and 104, and cinching the straps 94 and 100 as necessary.

With the base panel 82 so disposed, a prosthetic phallus 200 can then be engaged with the retention panel 84 by peeling the retention panel 84 at least partially away from the base panel 82 and passing the shaft portion 202 of the phallus 200 through the phallus aperture 86 as shown in FIG. 20. Then, the retention panel 84 can be engaged with the base panel 82 in a desired location and orientation, and the phallus 200 will be secured in place by the fastening means 112.

The retention panel 84 can be further secured in relation to the base panel 82 and the chair 400 by a belt structure 110 with a proximal end fixed to an end of the retention panel 84 and a buckle member 108 slidably engaged therewith in combination with a buckle member 106 fixed to an opposite end of the retention panel 84. Under this arrangement, the ends of the belt structure 110 can be passed under the seat 402 of the chair 400, the buckle members 106 and 108 can be engaged, and the belt 110 can be cinched as necessary to yield the arrangement shown in FIG. 20 with the retention panel 84 and the phallus 200 securely retained relative to the seat 402. Stimulus aids, such as bullet vibrating devices 208 can be retained in selective locations and orientations by use of some or all of the sleeves 88, 90, 92, and 118.

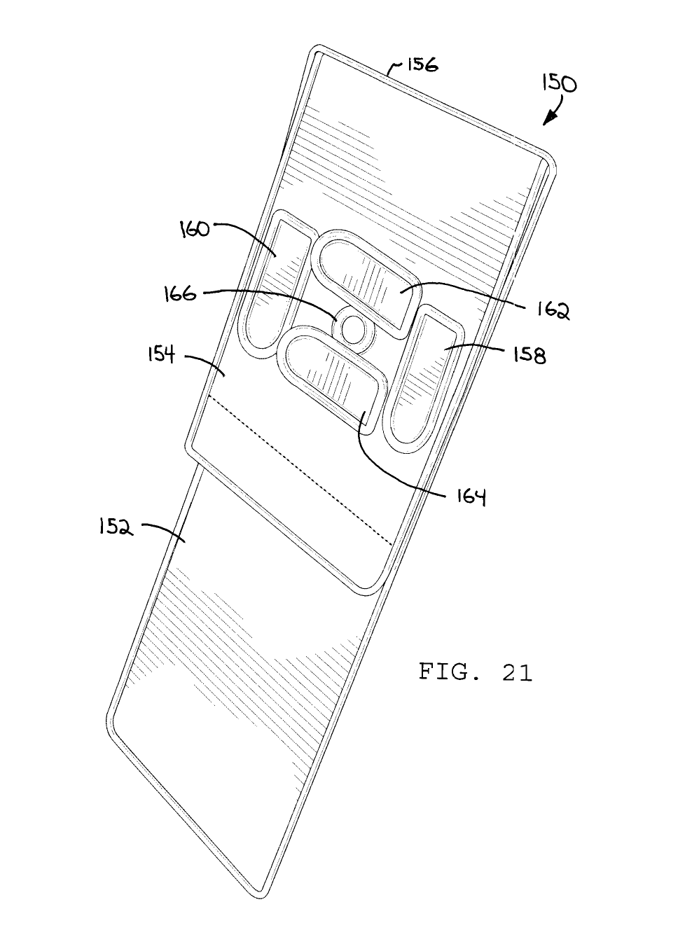

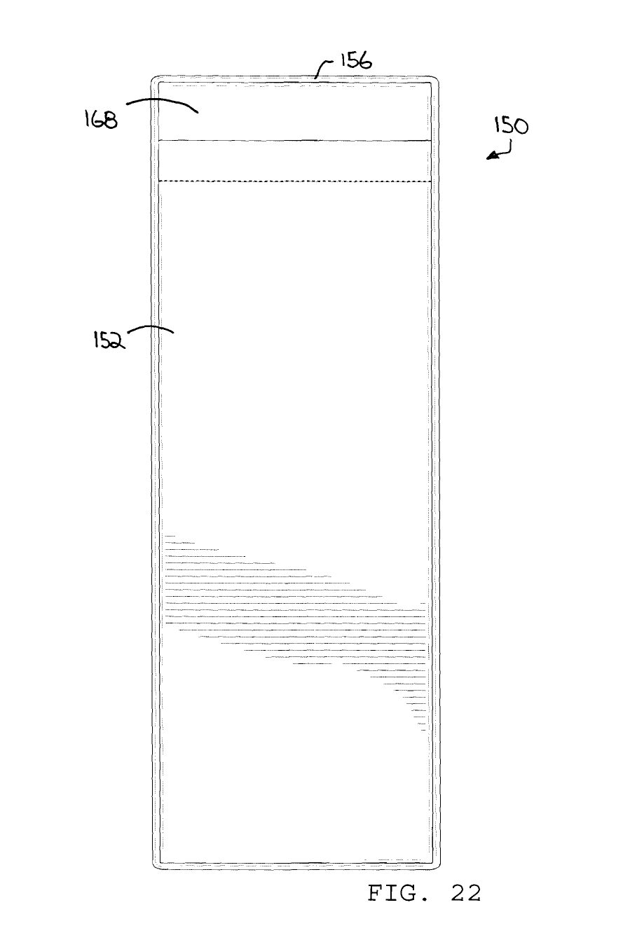

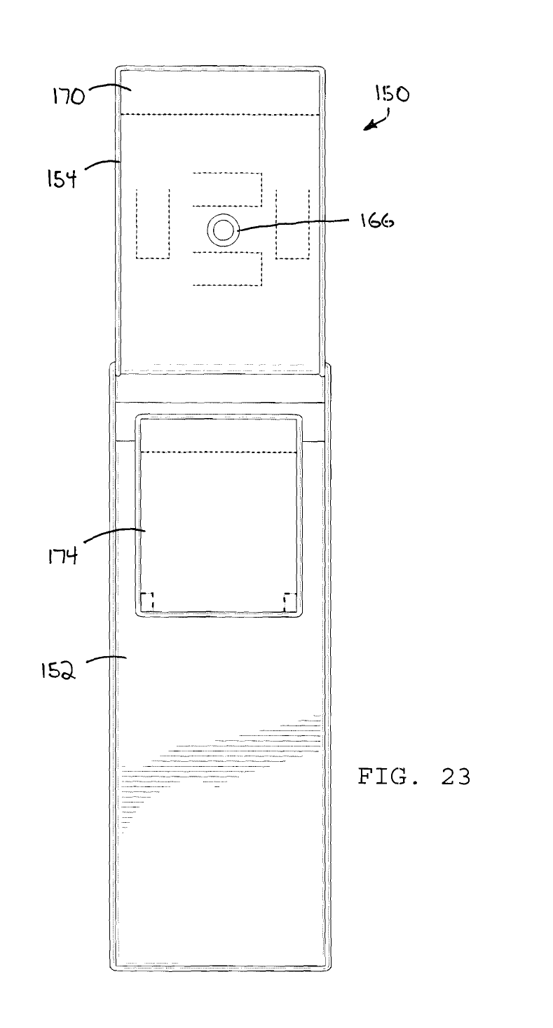

A further phallus retention apparatus taking advantage of the present invention is indicated generally at 150 in FIGS. 21 through 25. In this embodiment, the phallus retention apparatus 150 is founded on a base panel 152. As can be appreciated by reference to FIG. 22, the base panel 152 has a fastening means 168 applied to a first end thereof for selectively engaging the outer surface of the base panel 152 to form the base panel 152 into a loop. The fastening means 168 could comprise a hook and loop combination, mechanical fastening tape, or any other effective means. In a presently contemplated embodiment, the base panel 152 is formed from neoprene, and the fastening means 168 comprises a mechanical fastening tape that can selectively fasten to the outer surface of the base panel 152. Of course, numerous other fastening means 168 would be possible and within the scope of the invention. So constructed, the base panel 152 can be looped around a given structure, such as a person's thigh 500 as shown in FIG. 25, another extremity, or any other structure.

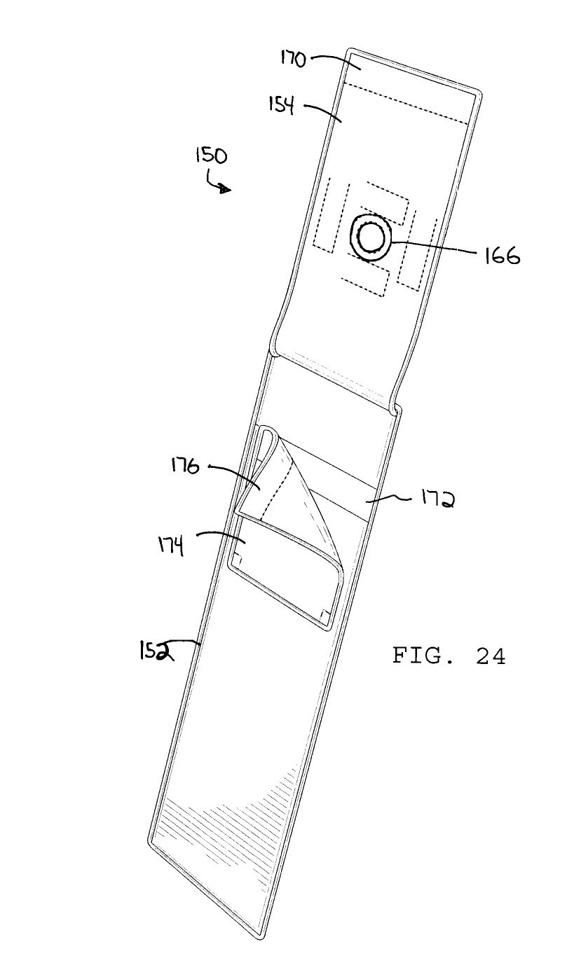

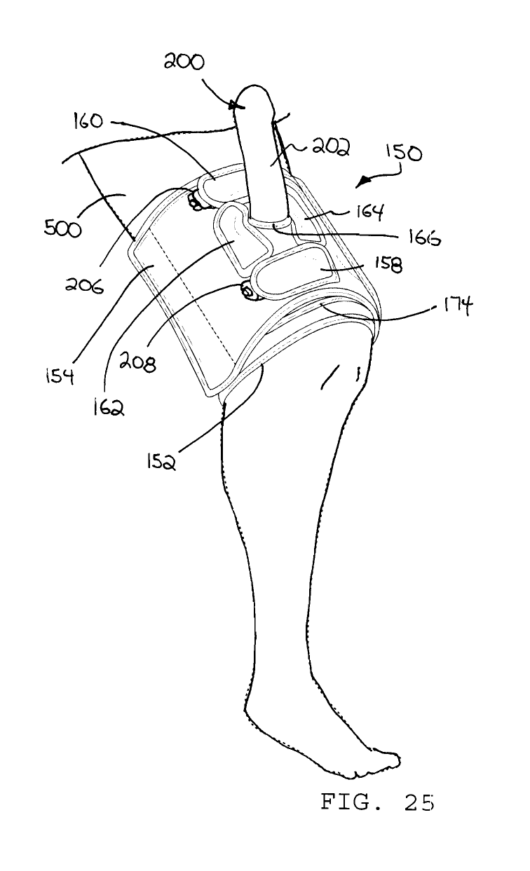

A retention flap 154 has a proximal end secured, such as by sewing, integral formation, or otherwise, to an end of the base panel 152 to establish a hinge connection 156. The retention flap 154 has a phallus aperture 166 defined by a resiliently flexible ring centrally disposed therein. A fastening means 170, such as a mechanical fastening tape that selectively fastens to the outer surface of the base panel 152, is disposed adjacent to the distal end of the retention flap 154. With this, the flap 154 can be pulled to the open configuration shown in FIGS. 23 and 24 to enable the shaft portion 202 of a prosthetic phallus 200 to be passed through the phallus aperture 166. With the phallus 200 so disposed, the flap 154 can be selectively secured in position so that the phallus 200 will be securely retained as in FIG. 25.

Sleeves 158, 160, 162, and 164 can be disposed adjacent to the aperture 166 for receiving and retaining vibratory devices 206 and 208 or other stimulus aids. In certain embodiments, one or more of the sleeves 158, 160, 162, and 164 can be removably attached to the retention flap 154, such as by a hook and loop combination or mechanical retention tape. With this, the number, location, and orientation of the vibratory devices 206 and 208 can be adjusted to suit a given user's preferences. As shown in FIG. 23, a further padding panel 174 can be removably coupled to the base panel 152 by mechanical fastening tape 176 or any other effective means to provide any necessary padding to the user by being interposed between the base portion 204 of the phallus 200 and the body of the user.

Under this construction, a person could make use of the phallus retention apparatus 150 by first peeling the retention flap 154 at least partially away from the base panel 152. Then, the body portion 202 of a phallus 200 can be inserted through the phallus aperture 166. The retention flap 154 can then be re-adhered to the base panel by use of the fastening means 170.

One or more stimulus aids, such as vibratory devices 206 and 208, can then be inserted into the sleeves 158, 160, 162, and 164, and the sleeves 158, 160, 162, and 164 can be positioned as desired. The base panel 152 can then be formed into a loop encircling a given body part or structure, such as a wearer's thigh 500 as shown in FIG. 25, and the fastening means 168 can retain the base panel 152 and the phallus retention apparatus 150 in general securely in place relative to the thigh 500 or other structure.

With certain details of the present invention for phallus retention apparatuses 10, 80, and 150 disclosed, it will be appreciated by one skilled in the art that changes and additions could be made thereto without deviating from the spirit or scope thereof. This is particularly true when one bears in mind that the presently preferred embodiments merely exemplify the broader invention revealed herein. Accordingly, it will be clear that those with certain major features of the claimed invention in mind could craft embodiments that incorporate those major features while not incorporating all of the features included in the preferred embodiments.

Therefore, the following claims are intended to define the scope of protection to be afforded to the inventor. Those claims shall be deemed to include equivalent constructions insofar as they do not depart from the spirit and scope of the invention. A plurality of the following claims may express certain elements as means for performing a specific function, at times without the recital of structure or material. As the law demands, these claims shall be construed to cover not only the corresponding structure and material expressly described in this specification but also all equivalents thereof that might be now known or hereafter discovered.

1. A phallus retention apparatus for retaining a prosthetic phallus relative to a hand of a wearer, characterized in that wherein the phallus retention apparatus comprises:
a sheath of flexible material wherein the sheath has a first end and a second end wherein the first end is open for receiving the hand of the wearer from a first direction;
a thumb aperture in the sheath through which a thumb of the wearer can be passed;
a prosthetic phallus aperture for receiving a shaft portion of a prosthetic phallus;
means for retaining the prosthetic phallus aperture relative to the sheath spaced from the thumb aperture.

2. The phallus retention apparatus of claim 1 wherein the material of the sheath is substantially continuous but for the thumb aperture.

3. The phallus retention apparatus of claim 1 wherein the second end of the sheath is open for receiving the hand of the wearer from a second direction whereby the phallus retention apparatus can be retained relative to the left or right hand of the wearer.

4. The phallus retention apparatus of claim 3 wherein the phallus aperture is disposed in the sheath.

5. The phallus retention apparatus of claim 4 wherein the sheath has a palmar side and a dorsal side and wherein the phallus aperture is spaced from the thumb aperture to position the phallus aperture approximately centered on the palmar side of the sheath.

6. The phallus retention apparatus of claim 5 wherein the phallus aperture is disposed approximately perpendicular to the thumb aperture.

7. The phallus retention apparatus of claim 6 wherein the phallus aperture and the thumb aperture are generally aligned laterally in relation to the sheath.

8. The phallus retention apparatus of claim 4 further comprising at least one cinching strap laterally retained relative to the sheath in combination with a means for cinching the cinching strap about the sheath thereby to adjust the effective size of the sheath to accommodate hands of different sizes.

9. The phallus retention apparatus of claim 4 further comprising at least one sleeve coupled to the sheath adjacent to the phallus aperture for selectively retaining a stimulus aid.

10. The phallus retention apparatus of claim 1 further comprising at least one finger loop is retained adjacent to the second end of the sheath for receiving one or more fingers of the wearer.

11. The phallus retention apparatus of claim 10 further comprising an extension panel with a proximal end retained by the sheath and wherein the at least one finger loop is retained by a distal end of the extension panel.

12. The phallus retention apparatus of claim 1 characterized in further comprising a retention flap with a proximal end fixed to the sheath, an elongate body portion with a distal end, and a means for selectively securing the retention flap wrapped at least partially around the sheath and wherein the phallus aperture is disposed in the retention flap.

13. The phallus retention apparatus of claim 12 wherein the sheath has a palmar side and a dorsal side and wherein the proximal end of the retention flap is fixed to the sheath adjacent to the palmar side of the thumb aperture.

14. The phallus retention apparatus of claim 12 wherein the sheath has a palmar side and a dorsal side and wherein the phallus aperture is spaced from the thumb aperture to position the phallus aperture approximately centered on the palmar side of the sheath when the retention flap is wrapped at least partially around the sheath.

15. The phallus retention apparatus of claim 14 wherein the phallus aperture is disposed approximately perpendicular to the thumb aperture when the retention flap is wrapped at least partially around the sheath.

16. The phallus retention apparatus of claim 15 wherein the phallus aperture and the thumb aperture generally align laterally in relation to the sheath when the retention flap is wrapped at least partially around the sheath.

17. The phallus retention apparatus of claim 14 further comprising at least one sleeve coupled to the retention flap adjacent to the phallus aperture for selectively retaining a stimulus aid.

18. The phallus retention apparatus of claim 1 further comprising at least one flexible ring with a central aperture for being passed over the shaft portion of the prosthetic phallus.

19. A phallus retention apparatus for retaining a prosthetic phallus relative to an external structure, wherein the phallus retention apparatus comprises:
a base panel;
a retention panel;
means for coupling the retention panel relative to the base panel;
a phallus aperture in the retention panel for receiving the body portion of the phallus therethrough; and
means for retaining the base panel and the retention panel in relation to the external structure.

20. The phallus retention apparatus of claim 19 wherein the means for fastening the retention panel to the base panel comprises mechanical fastening tape.

21. The phallus retention apparatus of claim 20 wherein the mechanical fastening tape entirely surrounds the resilient phallus aperture.

22. The phallus retention apparatus of claim 20 further comprising at least one sleeve and means for selectively retaining the at least one sleeve adjacent to the phallus aperture for selectively retaining a stimulus aid.

23. The phallus retention apparatus of claim 22 wherein the means for selectively retaining the at least one sleeve adjacent to the phallus aperture comprises mechanical fastening tape.

24. The phallus retention apparatus of claim 20 wherein the means for retaining the base panel and the retention panel in relation to the external structure comprises a means for selectively coupling a first end portion of the base panel to a second end portion of the base panel to surround at least a portion of the external structure.

25. The phallus retention apparatus of claim 24 wherein the means for selectively coupling the first end portion of the base panel to the second end portion of the base panel to surround at least a portion of the external structure comprises a means for directly coupling the first and second end portions of the base panel.

26. The phallus retention apparatus of claim 24 wherein the means for selectively coupling the first end portion of the base panel to the second end portion of the base panel to surround at least a portion of the external structure comprises at least one strap and means for securing the at least one strap to surround at least a portion of the external structure.

27. The phallus retention apparatus of claim 20 wherein the retention panel and the base panel are separable.

28. The phallus retention apparatus of claim 20 wherein the retention panel and the base panel have first ends secured together at a hinge connection.

29. The phallus retention apparatus of claim 20 wherein the phallus retention apparatus further comprises at least one sleeve and means for retaining the at least one sleeve adjacent to the phallus aperture for selectively retaining a stimulus aid.

30. The phallus retention apparatus of claim 29 wherein the means for retaining the at least one sleeve comprises a means for selectively retaining the at least one sleeve in a desired position and orientation.
